(19) United States
(12) Patent Application Publication
(10) Pub. No.: US 2006/0201989 A1
(43) Pub. Date: Sep. 14, 2006

(54) Surgical anvil and system for deploying the same

(76) Inventor: Herminio F. Ojeda, San Antonio, TX (US)

Correspondence Address: NEEDLE & ROSENBERG, P.C., SUITE 1000, 999 PEACHTREE STREET, ATLANTA, GA, 30309-3915, US

(21) Appl. No.: 11/375,355

(22) Filed: Mar. 13, 2006

Related U.S. Application Data
(60) Provisional application No. 60/660,852, filed on Mar. 11, 2005.

(51) Int. Cl. A61B 17/10

(52) U.S. Cl. 227/175.1

(57) ABSTRACT

A system for deploying an anvil in a subject comprises a tube having a first open end, a second open end and a lumen that extends between the respective first and second ends. The system further comprises an anvil configured to selectively move between a compressed and an expanded position. In one aspect, in the expanded position, at least a portion of the anvil is configured to form staples fired by the surgical stapling device after the anvil is expelled from the second open end of the tube.

### CROSS-REFERENCE TO RELATED APPLICATIONS

This application claims the benefit of U.S. Provisional Application No. 60/660,852, filed on Mar. 11, 2005, which is herein incorporated by reference in its entirety.

### BACKGROUND

Obesity is an epidemic and decreases life expectancy and quality of life. The National Institutes of Health have estimated that only 5% of medically supervised weight loss programs are successful in the long term. Over the years, surgical operations have been used to successfully assist patients in controlling their weight. In particular, gastric bypass surgery has provided the only means of long term weight loss to many patients.

The gastric bypass operation is designed to limit the amount of food a patient consumes. This is done by creating a pouch from the patient's stomach that only holds a small amount of food. The pouch is connected directly to the patient's small intestine through a surgical anastomosis. Consumed food moves through the pouch and into the small intestine and bypasses the majority of the patient's stomach. Because the pouch is so small, a small amount of food satiates the patient leading to long term weight loss.

The surgical anastomosis created between the gastric pouch and the intestine is usually accomplished by stapling the intestine to the pouch. A surgeon typically uses a circular stapler to fire the staples, which requires placement of a circular stapler anvil in the pouch. The anvil guides the placement of the stapler and forms the staples to accomplish the anastomosis. Placement of the anvil is one of the most time consuming and difficult parts of the gastric bypass operation.

Current methods of anvil placement include laparoscopic and transoral procedures. Current laparoscopic methods require excessive suturing and transoral methods can cause life threatening esophageal injury. Moreover, these methods are time consuming, requiring significant anesthesia and operation time, and can have high wound infection rates. These current methods may further require the services of a gastroenterologist for evaluation of the anastomosis site and may require a surgeon to break scrubs, causing an increase in the time and cost of performing the gastric bypass procedure.

### SUMMARY

In one embodiment of the present invention, a system is provided for deploying an anvil useable with a surgical stapling device. In one aspect, the system comprises a tube and an anvil that is configured to slide though a lumen of the tube. In one exemplary aspect, the anvil comprises a shaft member with a distal portion configured for engagement with the stapling device and a head member. In one aspect, the head member is moveable between an intraluminal position and an extraluminal position. In the intraluminal position, the head member is configured for slideable movement of the anvil thorough the lumen of the tube. In the extraluminal position, at least a portion of the head member is configured to form staples fired by the surgical stapling device after the anvil is expelled from an open end of the tube.

The described system can be used to transorally place an anvil during gastric bypass surgery in a subject. The described system can also be used to place an anvil during other operations in a subject including, but not limited to, colo-colonic anastomosis during colon resections, small bowel resections and gastro-intestinal anastomosis.

Other apparatus, methods, and aspects and advantages of the invention will be discussed with reference to the Figures and to the detailed description of the preferred embodiments.

### BRIEF DESCRIPTION OF THE DRAWINGS

The figures are not necessarily drawn to scale, emphasis instead being placed upon illustrating the principles of the invention. The accompanying drawings, which are incorporated in and constitute a part of this specification, illustrate certain aspects of the instant invention and together with the description, serve to explain, without limitation, the principles of the invention. Like reference characters used therein indicate like parts throughout the several drawings.

FIG. 1A is a side elevational view of a first embodiment of an anvil of the present invention in an intraluminal compressed position.

FIG. 1B is a side elevational view of the first embodiment of the anvil of the present invention in an extraluminal expanded position.

FIG. 2A is a side elevational view of a second embodiment of an anvil of the present invention in an intraluminal compressed position.

FIG. 2B is a side elevational view of the second embodiment of the anvil of the present invention in an extraluminal expanded position.

FIG. 3 is a perspective view of the anvils shown in FIGS. 1B and 2B.

FIG. 4A is a side perspective view of a third embodiment of an anvil of the present invention in an intraluminal compressed position.

FIG. 4B is a side elevational view of the third embodiment of the anvil of the present invention in an extraluminal expanded position.

FIG. 5A is a side perspective view of a fourth embodiment of an anvil of the present invention in an intraluminal compressed position.

FIG. 5B is a side elevational view of the fourth embodiment of the anvil of the present invention in an extraluminal expanded position.

FIG. 6 is a perspective view of the anvils shown in FIGS. 4B and 5B.

FIG. 7A is a side elevational view of a fifth embodiment of an anvil of the present invention in an intraluminal compressed position.

FIG. 7B is a side perspective view of the fifth embodiment of the anvil of the present invention in an extraluminal expanded position.

FIG. 8A is a side elevational view of a sixth embodiment of an anvil of the present invention in an intraluminal compressed position.

FIG. 8B is a side perspective view of the sixth embodiment of the anvil of the present invention in an extraluminal expanded position.

FIG. 9 is a perspective view of the anvils shown in FIGS. 7B and 8B.

FIG. 10 is a schematic view showing an embodiment of a system of the present invention for deploying an anvil.

FIG. 11 is a schematic view showing an embodiment of a system of the present invention for deploying an anvil.

FIG. 12 is a perspective view of a seventh embodiment of the anvil of the present invention in an extraluminal expanded position.

FIG. 13A is a side perspective view of the anvil of FIG. 12 in an intraluminal compressed position.

FIG. 13B is a side elevational view of the anvil of FIG. 12 in the extraluminal expanded position.

FIG. 14 is a perspective view of an eighth embodiment of the anvil of the present invention in an extraluminal expanded position.

FIG. 15 is a side view of a head of an anvil that is pivotally movable relative to the longitudinal axis of the shaft of the anvil.

### DETAILED DESCRIPTION OF THE INVENTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to “an alignment member” includes aspects having two or more such alignment members unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not. For example, the phrase “optionally comprises a sizing balloon” means that the system may comprise a sizing balloon or may not comprise a sizing balloon such that the description includes both the sizing balloon and the absence of the sizing balloon.

As used in the specification and the appended claims, by a “subject” is meant an individual. The term does not denote a particular age or sex. In one aspect, the subject is a mammal such as a primate, including a human. The term includes human and veterinary subjects.

In one embodiment of the present invention, provided herein is a system for deploying an anvil in a subject. In one embodiment the system comprises a tube having an open first end, an open second end and a lumen that extends between the respective first and second ends. The system further comprises an anvil configured for use with a surgical stapler that is selectively positionable within the lumen. Optionally, the tube is an orogastric tube, which can optionally include a sizing balloon for sizing a gastric pouch in the subject. Of course, the tube can range in size as desired. For example and not meant to be limiting, the tube can be 20 to 50 French (fr) in size.

In one exemplified use, the anvil is moveable through the lumen of the tube and the system can further comprise an anvil advancing member. In one aspect, the anvil advancing member moves the anvil through the lumen in a direction from the first open end towards the second open end. Optionally, the anvil advancing member is a plunger analogous to the plunger of a common syringe. In another aspect, the plunger can comprise an anvil advancing portion, which is the portion of the plunger that contacts the anvil, or an intermediate force transferring member to selectively push or urge the anvil through the lumen of the tube. Thus, in one aspect it is contemplated that the anvil advancing portion of the plunger is moveable through the lumen of the tube.

In one aspect, the anvil used in the disclosed system comprises a shaft member and a head member. In this aspect, the shaft member comprises a distal end portion that is configured for mating with a receiving portion of a circular stapling device. In another aspect, the head member of the anvil is moveable between a compressed position having a reduced diameter and an expanded or deployed position. Optionally, the shaft member further comprises an alignment member. In a further aspect, the head member further comprises a staple forming surface that optionally includes a plurality of staple forming recesses or buckets defined therein. In one aspect, the staple forming buckets can be arranged in at least one concentric row about the staple forming surface. The staple forming surface can further comprise a deformable intermediate portion. In some aspects, the head member can comprise a plurality of struts.

In use, the head member is positioned within the lumen of the tube in its compressed or intraluminal position. In some aspects, as described below, the head member is compressed away from the shaft member. In other aspects, also described below, the head member is compressed towards the shaft member. In alternative aspect, the head member is configured to move from its extraluminal position to its intraluminal position by deflection of the head member toward the longitudinal axis of the shaft member of the anvil, and to move from its intraluminal position to its extraluminal position by deflection of the head member away from the longitudinal axis of the shaft member of the anvil. In a further aspect, the head member can further comprise a knife cutting surface.

In one aspect, the head member can comprise a material that has material memory. For example, the head member can be formed from Nitinol. The memory properties of the material can urge, or help urge, the head member from its compressed to its expanded or deployed position. In other aspects, a biasing mechanism can be used to move the head member from its compressed position to its expanded position. For example, a wire spring can be used.

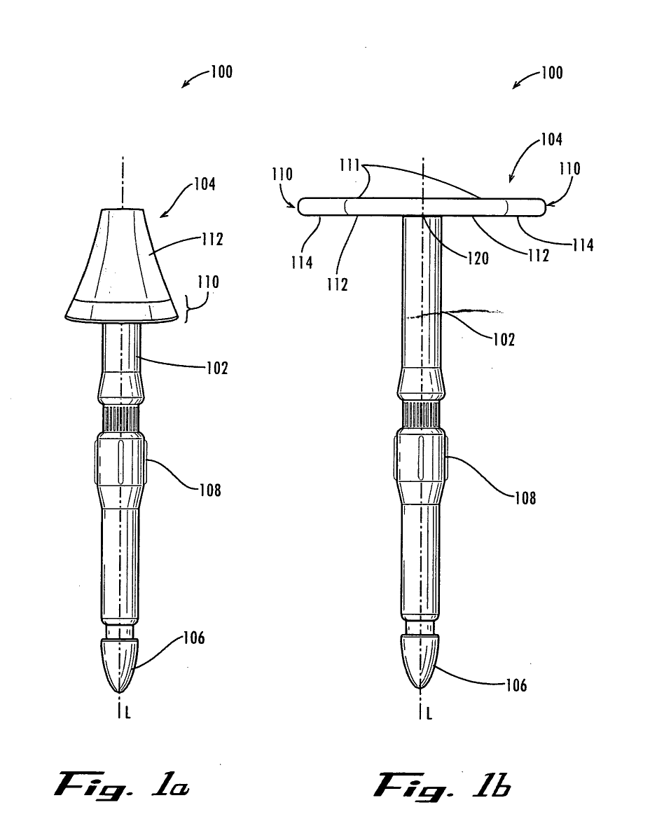

FIGS. 1A and 1B show a first embodiment of an anvil 100. The anvil comprises a shaft member 102 and a head member 104. The shaft member 102 has a proximal portion 120 and a distal portion 106. The head member 104 is connected to the proximal portion 120 of the shaft member 102. The distal portion 106 of the shaft member 102 is constructed and arranged to connect with an anvil connecting member of a circular stapler. This connection is conventionally made by a male to female union, with the distal shaft portion 106 being received into an anvil connecting member of a circular stapler.

Circular stapling instruments typically have a mechanism for firing a plurality of staples from a staple holding cartridge against an anvil disposed opposite the staple cartridge. Such instruments typically apply one or more concentric/circular rows of staples. In use, a subject's tissue is located between the staple cartridge and anvil, and, by firing the instrument, staples become clenched to the tissue.

Circular staplers are known and have been successfully used in surgical procedures for many years. Commercially available instruments include the CEEA®. circular stapler, manufactured by United States Surgical Corporation, Norwalk, Conn., and the ILS® circular stapler, manufactured by Ethicon, Inc., Blue Ash, Ohio. Various embodiments of circular staplers have been disclosed in U.S. Pat. Nos. 4,576,167, 4,603,693, 5,005,749, and 5,119,983. The conventional connection between a “female” anvil connecting member and a distal “male” shaft portion is illustrated in U.S. Pat. Nos. 5,758,814 and 6,053,390, which are incorporated herein in their entirety by reference.

Once the anvil shaft 102 is received into the anvil connecting member of the circular stapling instrument, the anvil 100 is drawn towards and underlying the stapling cartridge. The circular stapler is then fired to cause the staples to pass through the subject's tissue that are positioned between the anvil and the circular stapling instrument and become formed against the anvil 100. During the firing step, a circular knife is typically advanced to cut tissue inside the staple line, thereby establishing a passage through the subject's tissues. After firing, the instrument is usually removed by withdrawing the anvil through the formed passage.

In one aspect, the head member 104 has a proximal surface 111 and a distal surface 112. The head member 104 is moveable between a compressed, intraluminal position as shown in FIG. 1A, and a deployed or expanded, extraluminal position shown in FIG. 1B. In one aspect, as shown in FIG. 1A, in its compressed position, portions of the head member 104 are deflected towards the surface of the shaft member 102. In this aspect, when the head member 104 moves from the compressed position to the deployed or expanded position, portions of the head member 104 move outward and away from the surface of the shaft member to form a substantially planar structure.

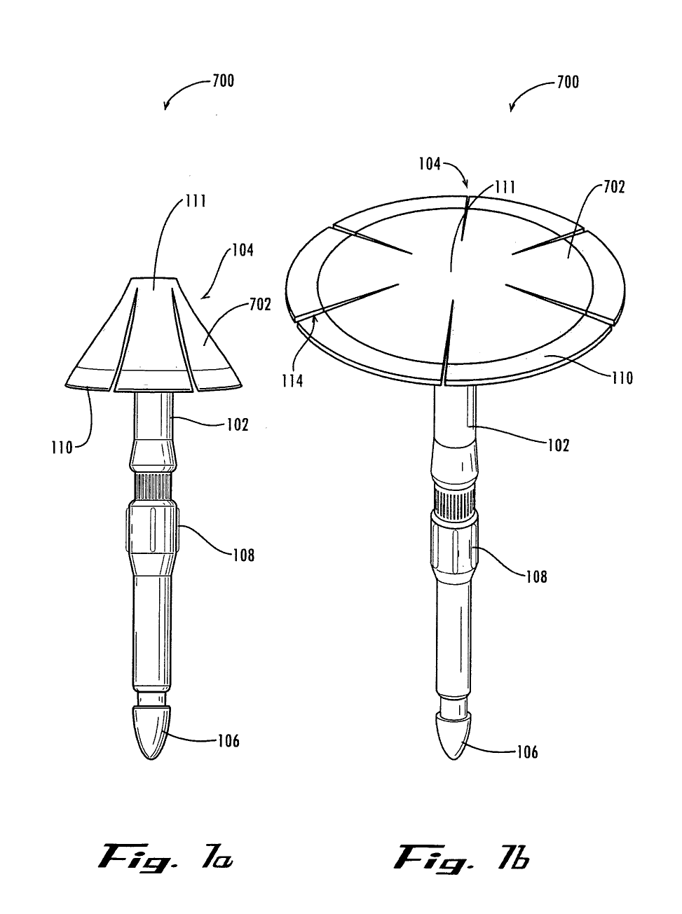

The head member 104 can comprise a concentric staple forming portion 110. In one aspect, the staple forming portion 110 is concentrically located proximate the periphery of the head member 104. At least a portion of the distal surface of the staple forming portion 110 comprises a staple forming surface 114. In one exemplary aspect, the staple forming surface 114 is generally made from a rigid material. However, in some aspects and as shown in FIG. 7, the distal surface 112 of the staple forming portion 110 comprises a plurality of rigid staple forming surfaces 604 separated by a plurality of deformable intermediate portions 602, as described in more detail below.

The head member 104 is typically compressed as shown in FIG. 1A so that the anvil 100 can be positioned inside the lumen 1004 of a tube 1002. In one aspect, the tube 1002 in which the anvil 100 is positioned is an orogastric tube and is approximately 30.0 French in size. In other aspects, for example, the tube or orogastric tube can be approximately 20.0 French to approximately 50.0 French in size. For example, an orogastric tube can be from about 40.0 French to about 42 French. The tube or orogastric tube can also vary in length. For example, the length of the tube or orogastric tube can be about 20.0 cm to about 100 cm in length or longer. Because the head member 104 is typically compressed against the luminal walls of the tube 1002 the size of the head member 104 in its compressed position typically varies depending of the size of the tube 1002 used. As one skilled in the art will recognize, the anvil head 104 can be compressed for manual or automated placement within the lumen 1004 of a tube 1002.

The diameter of the expanded head member 104 can vary depending on factors including, but not limited to, the model of circular stapler used, the size tube used, or upon surgical considerations as would be clear to one skilled in the art. In some aspects, in its expanded, extraluminal position, the diameter of the head member 104 can range from approximately 20.0 millimeters (mm) to about 35.0 mm. The diameter of the head in its extended position can be selected based on the surgery performed or on the site of desired anastomosis. For example, the diameter of the head member for gastric bypass can be about 21.0 mm, about 23.0 mm, about 25.0 mm or about 28.0 mm. In one aspect, the diameter of the expanded head member 104 is about 21.0 mm. The desired size of the head member, as well as the size of the tube, including is diameter and length, can be determined based on the teachings herein in combination with patient and/or surgical considerations that can be evaluated by one skilled in the art. For example and not meant to be limiting, the sizes used can depend upon a variety of factors including the disorder being treated and the severity of the disorder; the anastomosis site, the surgery performed; surgical and medical considerations including, the age, body weight, general health, sex and diet of the patient; and drugs used in combination or coincidental with the surgical device and like factors well known in the medical arts.

In another aspect, the shaft member 102 can further comprise an alignment member 108. An alignment member 108 can vary in shape and size and the shaft member 102 can also comprise a plurality of alignment members. As described above, after the distal shaft portion 106 is received into the anvil connecting member of a circular stapler, the anvil head 104 and the staple firing surface of the circular stapler are positioned and aligned such that a plurality of staples can be fired and formed against the staple forming surface 114 of the anvil head 104.

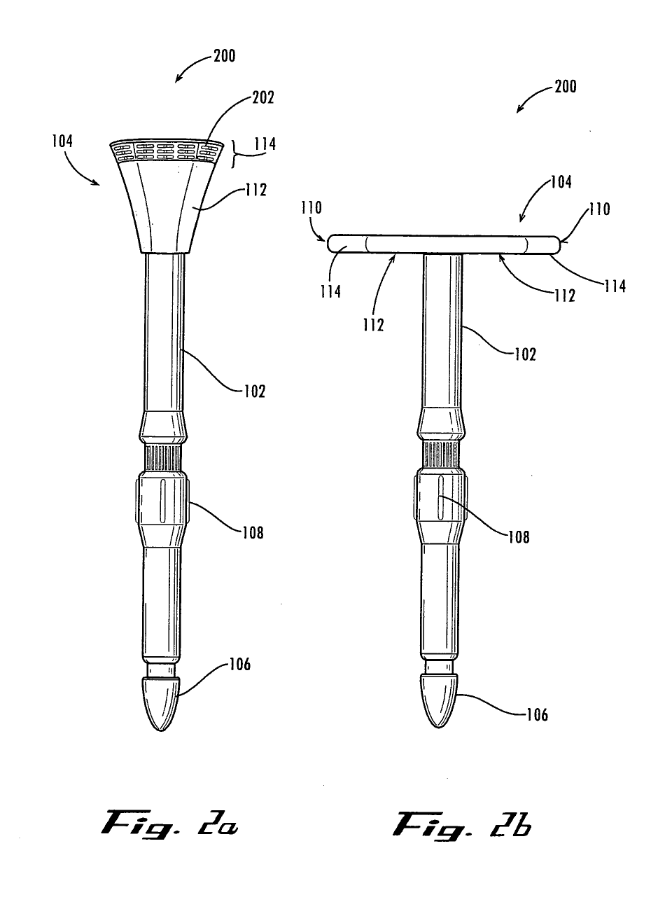

FIGS. 2A and 2B show a second embodiment of an anvil 200. In this embodiment, the head member 104 can be compressed for placement within the lumen 1004 of a tube 1002 by compressing portions of the head member 104 upwardly away from the anvil shaft 102 as shown in FIG. 2A. One would appreciate that the head member, in the compress position, is compressed toward the extended longitudinal axis of the anvil shaft. In this compressed position, the staple forming surface 114 can be visualized, and can comprise a plurality of crimping recesses 202 or “buckets” defined therein. When deployed and in operation, the recesses 202 clinch staples fired from the circular stapler against the staple forming surface 114 of the anvil head 104.

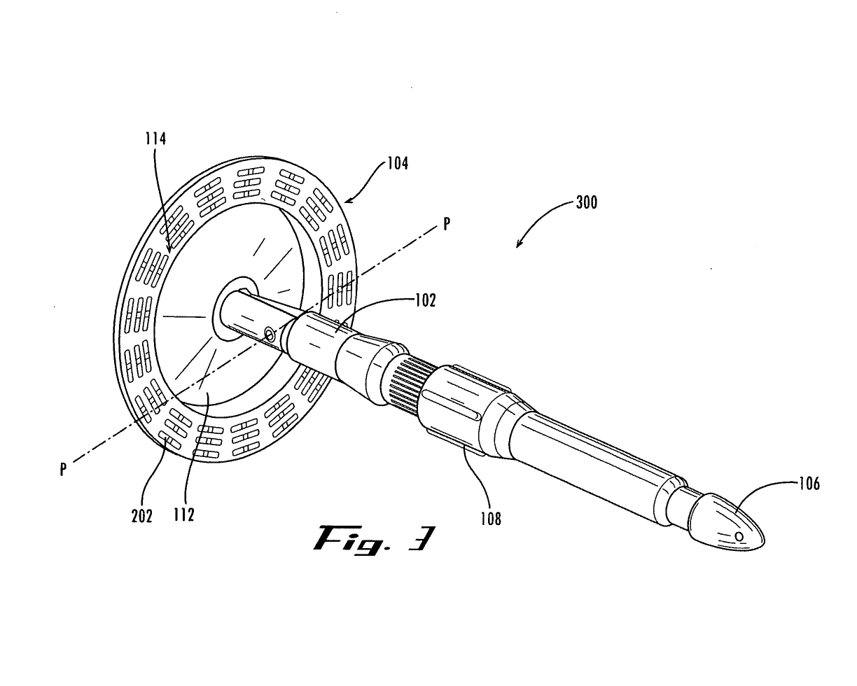

These buckets 202 for clinching staples are known in the art, and are better illustrated in FIG. 3, which shows a perspective view of the first and second embodiments of the anvil in the deployed or expanded, extraluminal position. In this aspect, the anvil head 104 has a concentric staple forming surface 114, which has the plurality of buckets 202 formed thereon. The buckets 202 are typically arranged in concentric rows about the staple forming surface 114. The number of bucket rows can vary depending on the circular stapling device used or on how many rows of staples are applied by the stapling device. For example, the staple forming surface 114 can comprise 1, 2, 3, 4, or more, rows of buckets 202. In one aspect, if a circular stapler fires 1 row of staples, then the staple forming surface typically has 1 row of buckets 202 to receive the staples when fired. As exemplified in FIG. 3, the staple forming surface 114 has three concentric rows of staple forming buckets 202. In this aspect, a circular stapler with three rows of staples is preferably used, but a stapler with one or two rows of staples can be used also.

In another aspect, at least a portion of the distal surface portion 112 of the anvil head 104 is located nearer to the shaft member 102 than the staple forming surface 114. This portion of the distal surface portion 112 can act as a knife cutting surface of approximation for a conventional circular knife of a circular stapler. In this aspect, the knife of a circular stapler can cut a subject's tissue that is interposed between the cutting edge of a circular knife and the distal surface portion 112 of the anvil head 104. During or after the firing of the circular stapler, a circular knife can be advanced to cut tissues inside the staple line. Because the circular knife cuts the subject's tissue inside the staple line, a passage is made through the subject's tissue. The passage is surrounded by the staples fired against the staple forming surface 114 forming an anastomosis.

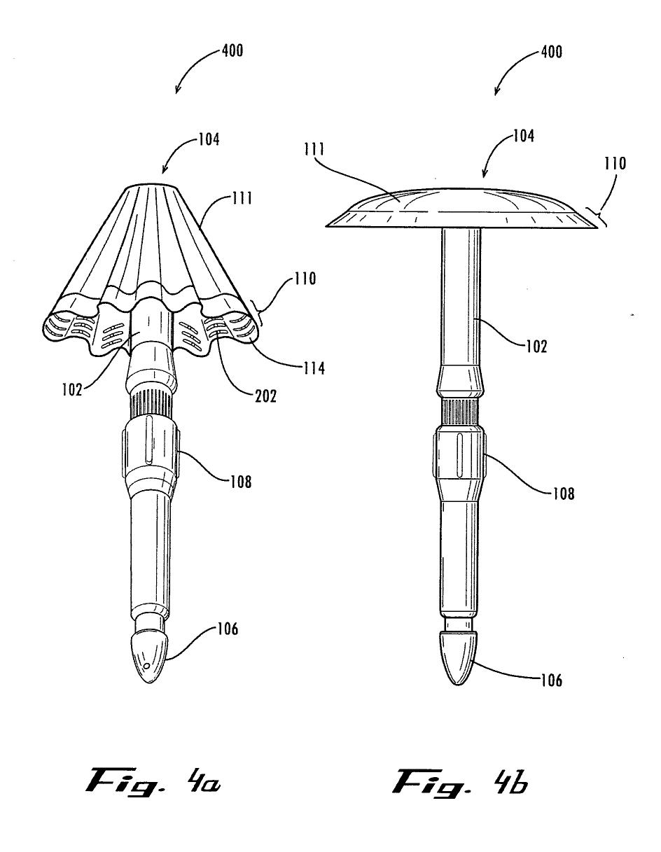

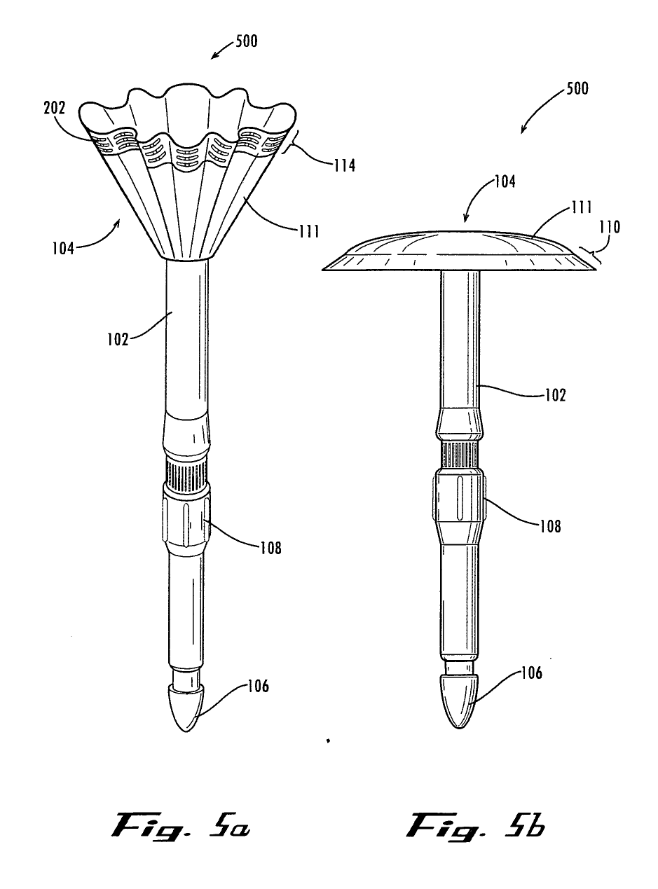

FIGS. 4A and 4B show a third embodiment of an anvil 400. The anvil head 104 is shown in a compressed, intraluminal position in FIG. 4A and in a deployed or expanded, extraluminal position in FIG. 4B. In its compressed position, portions of the anvil head 104 are compressed towards the surface of the shaft member 102. In its deployed position, the anvil head 104 is substantially dome shaped. As described above, the staple forming portion 110, comprises a staple forming surface 114 with staple forming buckets 202 on its distal surface. As shown in FIGS. 5A and 5B, in a fourth embodiment of the anvil, portions of the anvil head 104 can be compressed upwardly away from the shaft member 102.

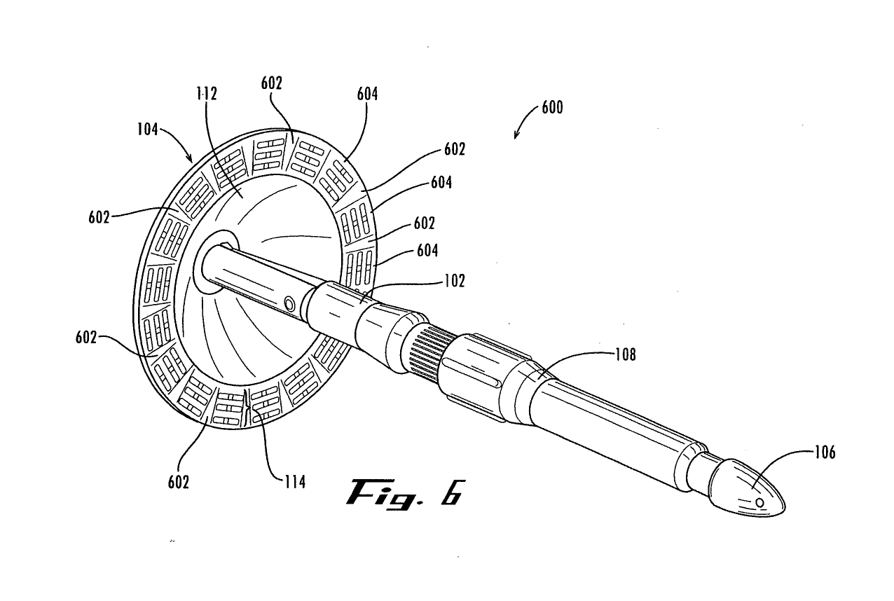

FIG. 6 shows a perspective view of the third and fourth embodiments of the anvil with its head member 104 in the deployed position. As exemplified in the illustration, the staple forming surface 114 can have a plurality of rigid portions 604 with staple forming buckets 202 formed thereon and a plurality of deformable intermediate portions 602. In one aspect, the deformable intermediate regions 602 are arranged adjacent to each rigid staple forming region 604. As one skilled in the art will appreciate, other arrangements of rigid staple forming regions 604 and deformable intermediate portions 602 are contemplated. For example, a first rigid staple forming region 604 may be adjacent to one or more staple forming region 604. In one aspect, adjacent rigid staple forming regions 604 are arranged adjacent to one or more deformable intermediate regions.

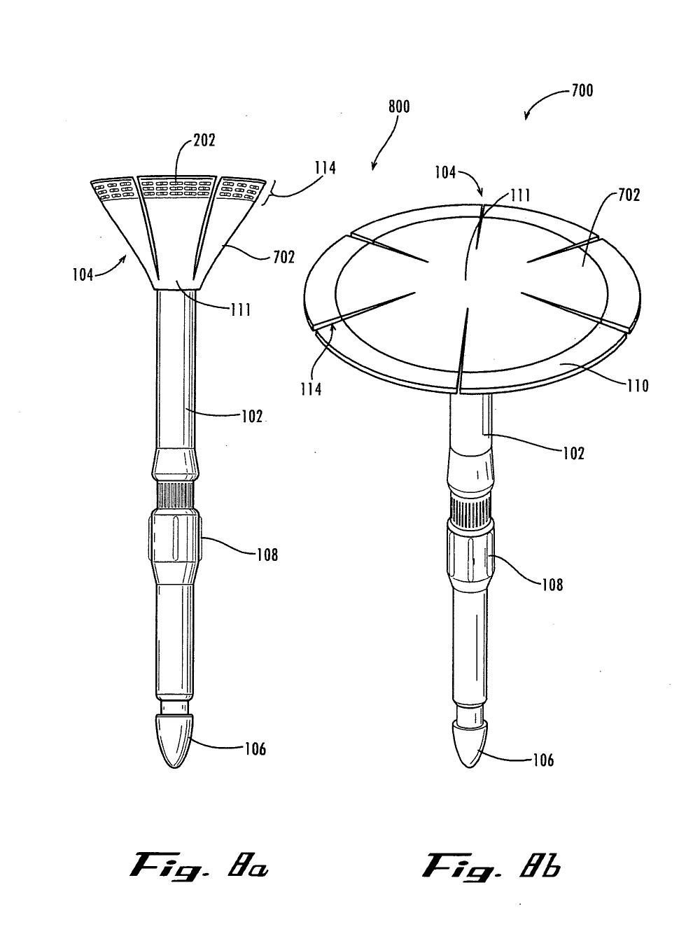

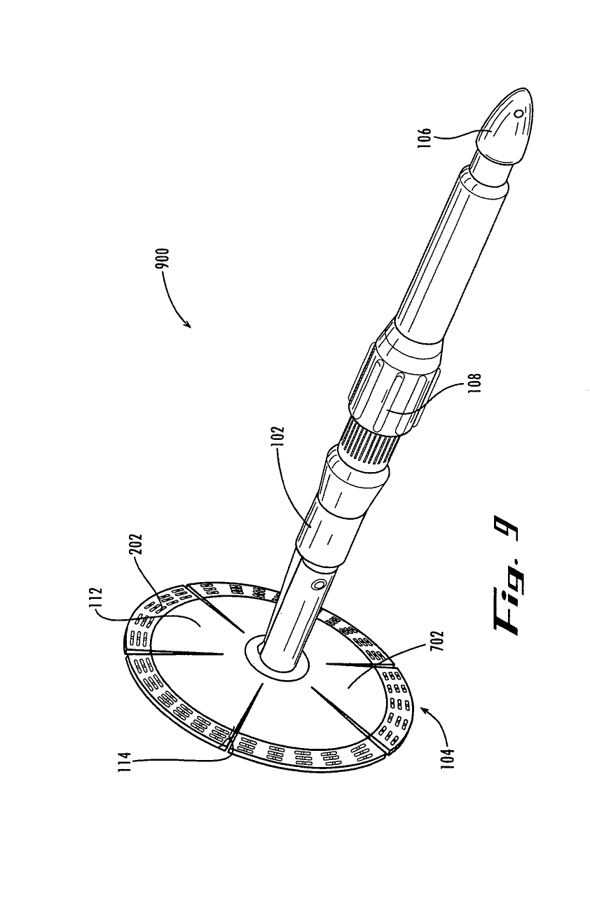

FIGS. 7A and 7B show a fifth embodiment of an anvil 700. In one aspect, the anvil head 104 of the anvil 700 comprises a plurality of strut members 702. Each strut member can further have a staple forming portion 110 with a staple forming surface 114. The plurality of struts 702 can be compressed towards the shaft member 102 and are moveable between the compressed position as shown in FIG. 7A and a deployed or expanded position as shown in FIG. 7B. In one aspect, the anvil head 104 comprises six strut members 702. In another aspect, the anvil head 104 comprises four strut members 702. As shown in FIG. 8A, in a sixth embodiment of the anvil 800, the strut members 702 can be compressed upwardly and away from the shaft member 102. FIG. 9 shows a perspective view of the fifth and sixth embodiments of the anvil in the deployed or expanded position. At least a portion of the strut members 702 of the anvil head 104 has a staple forming surface 114 that can comprise a plurality of staple forming buckets 202, which can be arranged in rows. The anvil head 104 strut members 702 can also have a distal surface portion 112 that is constructed and arranged to be contacted by a circular knife of a conventional circular stapler as described above.

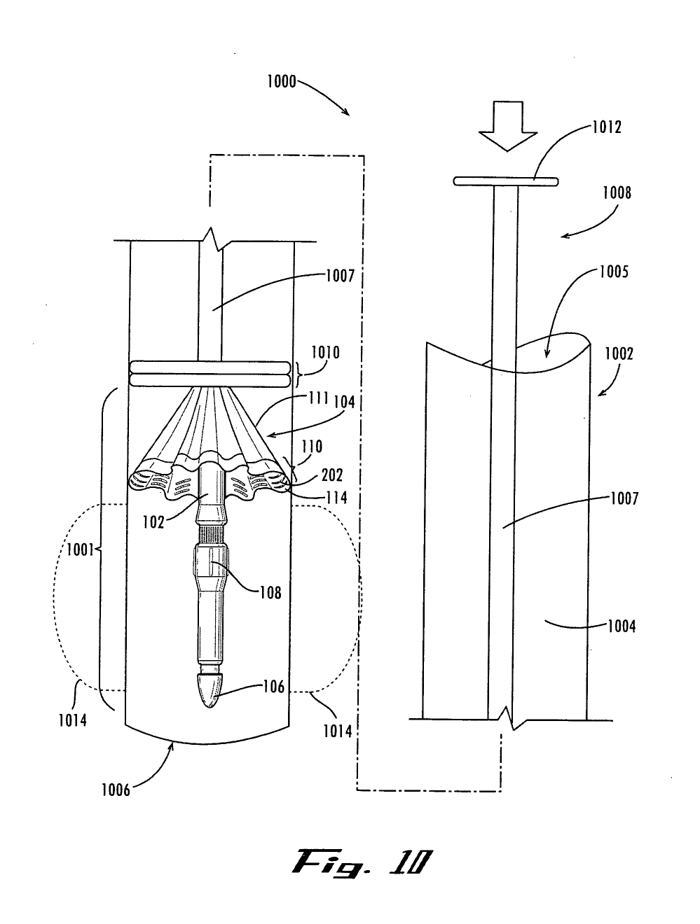

FIG. 10 shows an exemplary system 1000 of the invention. The system comprises an anvil 1001. As one will appreciate, it is contemplated that the anvil 1001 used can comprise any of the anvil embodiments described above. The system further comprises a tube 1002 with an open first end 1005, an open second end 1006 and a lumen 1004 that extends therebetween the first and second ends. In one aspect, the tube can be flexible, rigid, or semi-rigid. In one embodiment, the tube can be placed transorally into the subject's stomach. In a preferred embodiment, the tube is an orogastric tube. The tube 1002 is typically sized at 20-50 French, but other sizes can also be used.

The system can also comprise and anvil advancing member, such as, for example, a plunger 1008. The anvil advancing member is not limited to a plunger, however, and any advancing means can be used. For example, the anvil can be pulled through the tube lumen by an anvil advancing member such as a wire or suture material attached to the shaft portion of the anvil. The anvil can also be propelled through the lumen by pressurized gas or fluid. Thus, the anvil advancing member can be any mechanism that can be used to slidably move the anvil through the tube lumen. The anvil 1001 is positioned in its compressed position, within a portion of the lumen 1004 of the tube 1002.

In one aspect, the head member 104 of the anvil is held in its compressed, intraluminal position by the luminal walls of the tube 1002. When the anvil is expelled from the lumen 1004 of the tube 1002, the head member 104 typically expands to its expanded or deployed, extraluminal position. Of course, prior to insertion into the lumen 1004 of the tube 1002, the anvil head 104 can be in its expanded or deployed position. In this aspect, when inserted into the tube the head member 104 moves into its compressed position.

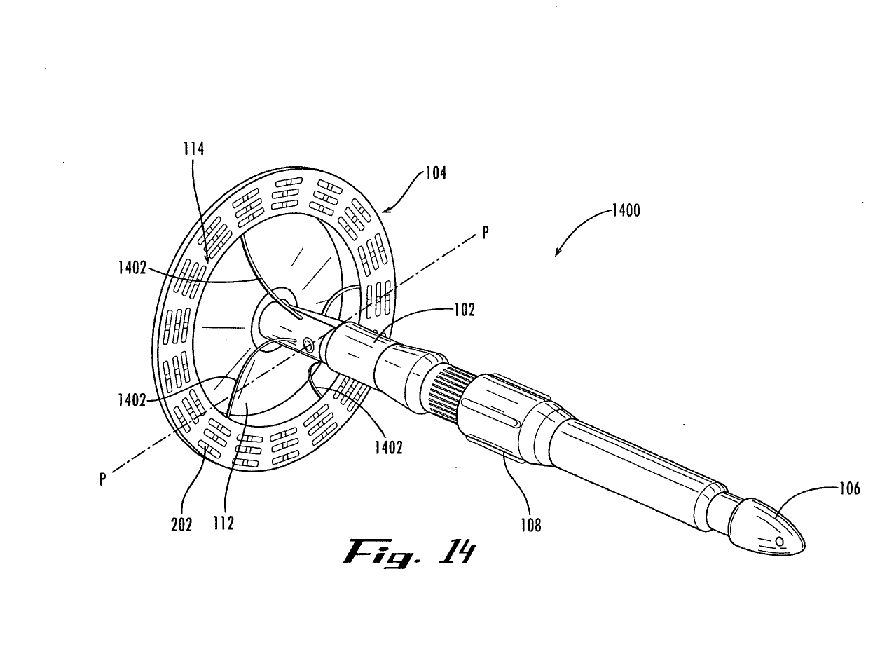

Movement of the head member 104 from its compressed, intraluminal position to its expanded or deployed, extraluminal position can be accomplished using biasing mechanisms known in the art. For example, the head member 104 can be optionally moved using a spring or plurality of springs to bias the head member 104 from its compressed to its expanded or deployed position. For example, a wire spring or a leaf spring, or a plurality of wire or leaf springs can be used. A perspective view of an eighth embodiment an anvil of the invention comprising a plurality of leaf springs 1402 is shown in FIG. 14. The leaf springs 1402 can be attached to the distal surface of the head member 112 and to the shaft 102. If a spring is used, the spring is typically loaded when the head member 104 is placed in its compressed position and the spring unloads causing the movement of the head member 104 to its deployed state upon expulsion from the lumen 1004.

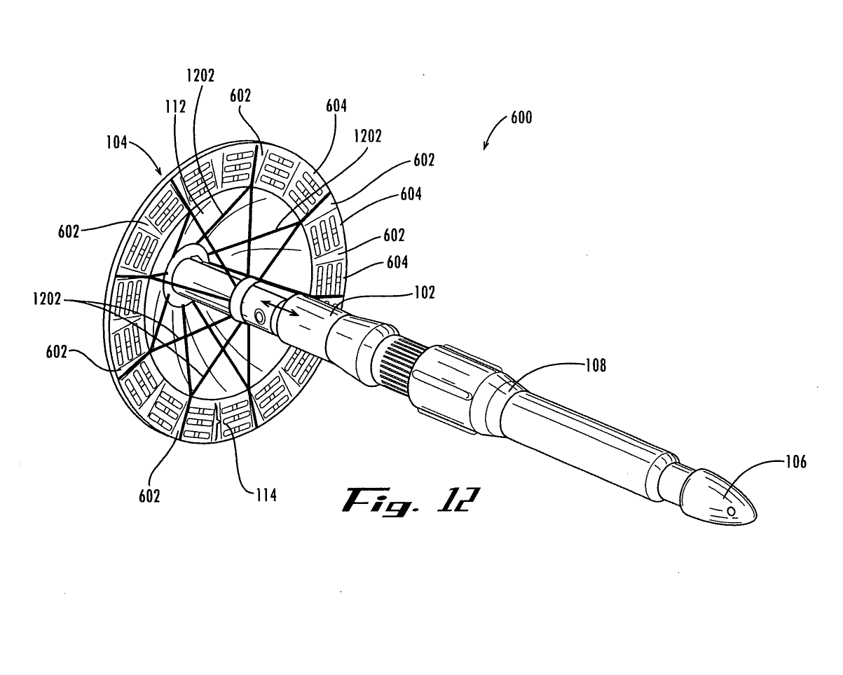

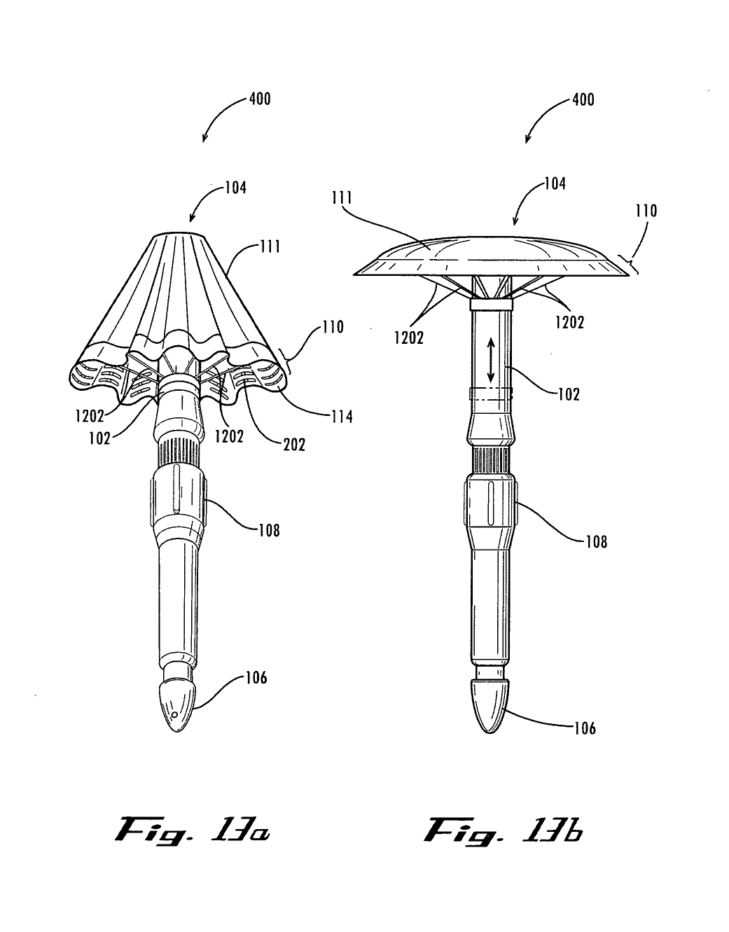

Other biasing mechanisms can also be used. For example, as shown in FIG. 12 and FIGS. 13A and 13B, the anvil can further comprise one or more rod 1202 analogous to the rods of a conventional umbrella. In this example, one end of a rod 1202 can be connectably attached to the distal surface 112 of the head member and the other end of the rod can be connectably attached about the shaft member 102 for slidable movement up and down the longitudinal axis of the shaft member. The rod about the shaft member can be slidably moved up and down the longitudinal axis of the shaft member, wherein upward movement of the rod causes expansion of the anvil head and downward movement of the rod causes compression of the head member about the shaft. The movement of the rod can be accomplished by a spring mechanism common to conventional umbrellas, or can be manually accomplished by the surgeon during the surgical procedure.

Thus, it is contemplated that the anvil described herein is not limited to the type of biasing mechanism employed. Any biasing mechanism capable of expanding the head member from its compressed to its deployed or expanded position can be used.

The attachment between the head member 104 and the proximal portion 120 of the shaft member 102 can be hinged or moveable such that the unloading force of a spring can bias the head member 104 into its expanded position. In aspects where the anvil head 104 is moved to its expanded position using a biasing mechanism such as a spring, the anvil head 104 can be formed from, for example, stainless steel, titanium alloy, tantalum alloy, plastics, or other materials known in the art and combinations thereof. The anvil shaft member 102 can also be made of these materials, and in one aspect is made of stainless steel.

The anvil head 104 can also move from its compressed position to its expanded position without using a mechanical biasing mechanism. For example, the head member can comprise a resilient material such as, for example, spring stainless steel, shape memory alloy, shape memory plastic, and the like. A head member 104 comprising these materials can be self expanding and can be constructed from a combination of these materials. Thus, the head member 104 can move to its expanded position when expelled from the lumen 1004 of the tube 1002. For example, the head member 104 can comprise Nitinol or other materials which have memory of an original expanded or deployed shape. When a self-expanding material is used, the head member's expanded or deployed shape is typically the shape that it will regain based on its memory characteristics.

In one aspect, the tube 1002 comprises a sizing balloon 1014. If used, the sizing balloon 1014 is located about the second end of the tube 1002. Thus, when the tube 1002 is placed in a subject, the sizing balloon 1014 can be used to size the gastric pouch during a gastric bypass procedure. The sizing balloon can be about 15 milliliters (ml) to about 100 ml in volume when inflated. In one aspect, the sizing balloon is approximately 30 milliliters (ml) in volume when inflated. The volume of the inflated sizing balloon 1014 can also be less than or greater than 15 ml or greater than 100 ml. For example, the inflated volume of the sizing balloon 1014 can be approximately 10 ml, 15 ml, 20 ml, 25 ml, 35 ml, 40 ml, 45 ml, 50 ml, 100 ml or greater. The sizing balloon can be inflated by an operator using a gas or fluid using methods known in the art. In one aspect, an additional lumen (not shown) can be used to inflate the sizing balloon.

In one aspect, the plunger 1008 comprises a proximal operating portion 1012, a distal anvil advancing portion 1010, and a shaft portion 1007 extending therebetween. In this aspect, proximal refers to the end of the plunger that would be nearest to the subject's mouth when the tube 1002 is placed in a subject. Similarly, distal refers to the end of the plunger that would be furthest from the subject's mouth when the tube 1002 is placed in a subject.

Thus, disclosed is a system 1000 for deploying an anvil 1001, and an anvil 1001, for use with a surgical stapling device. The system can comprise a tube 1002 having a first open end 1005, a second open end 1006 and a lumen 1004 extending therebetween the first and second ends. The system can further comprise an anvil 1001 for use with the surgical stapling device, the anvil having a shaft member 102 with a distal portion 106 configured for engagement with the stapling device and a head member 104. The head member is moveable between a first intraluminal position and a second extraluminal position. The intraluminal position is configured for slideable movement of the anvil 100 thorough the tube lumen 1004, and at least a portion of the extraluminal position is configured to form staples fired by the stapling device after the anvil is expelled from the lumen through the second open end.

The system can further comprise an anvil advancing means, such as a plunger 1008, for slideably moving the anvil though the lumen of the tube and for expelling the anvil through the second open end. If a plunger is used, the plunger can have a proximal operating portion 1012, a distal anvil advancing portion 1010 and a shaft portion 1007 extending therebetween. The plunger can be at least as long as the tube along the longitudinal axis of the tube.

The head member 104 can be configured to move between its extraluminal position and its intraluminal position by deflection of the distal surface 112 of the head member radially inward toward the shaft member 102 of the anvil, and to move between its intraluminal position and its extraluminal position by deflection of the distal surface 112 of the head member radially outward away from the shaft member 102 of the anvil. Biasing means can move the head member between its intraluminal position and its extraluminal position. The anvil head can also comprise material having shape memory for its extraluminal position such that the head moves between its intralumial position and its extraluminal position based on the shape memory of the material.

The head member 104 can also be configured to move between its extraluminal position and its intraluminal position by deflection of the proximal surface 111 of the head member radially toward the longitudinal axis of the shaft member 102 of the anvil at a position located proximal to the shaft member, and to move between its intraluminal position and its extraluminal position by deflection of the proximal surface 111 of the head member radially away from the longitudinal axis of the shaft member of the anvil at a position proximal to the shaft member. Biasing means can move the head member between its intraluminal position and its extraluminal position. The anvil head can also comprise material having shape memory for its extraluminal position such that the head moves between its intralumial position and its extraluminal position based on the shape memory of the material.

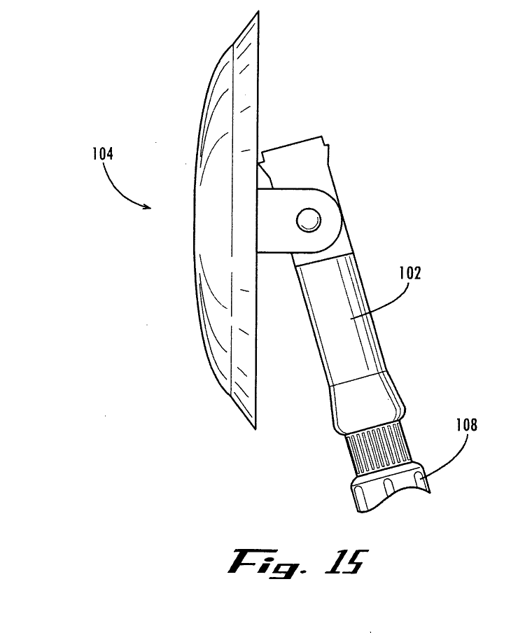

The head member 104 can also be pivotally moveable relative to the longitudinal axis of the shaft member. FIG. 15 is a side view of a head of an anvil that is pivotally moveable relative to the longitudinal axis of the shaft of the anvil. The head member can be pivoted about a pivot axis (p). The head member can be configured to move between its extraluminal position and its intraluminal position and between its intraluminal position and its extraluminal position by pivoting relative to the longitudinal axis, for example about the pivot axis (p), of the shaft member, wherein, in the extraluminal position, the head member is substantially perpendicular to the longitudinal axis of the shaft member, and in the intraluminal position, the head member is generally parallel to the longitudinal axis of the shaft member.

In practice, the anvil is positioned in the lumen between the second end of the tube and the anvil advancing portion. An operator can depress the proximal operating portion of the plunger 1012 in a distal direction towards the second end of the tube. By depressing the proximal operating portion of the plunger 1012, the distal anvil advancing portion 1010 of the plunger moves distally through the lumen 1004 of the tube 1002. The distal movement of the anvil advancing portion 1010 through the lumen 1004 of the tube 1002 advances the compressed anvil through the lumen 1004 in a distal direction towards the second end of the tube.

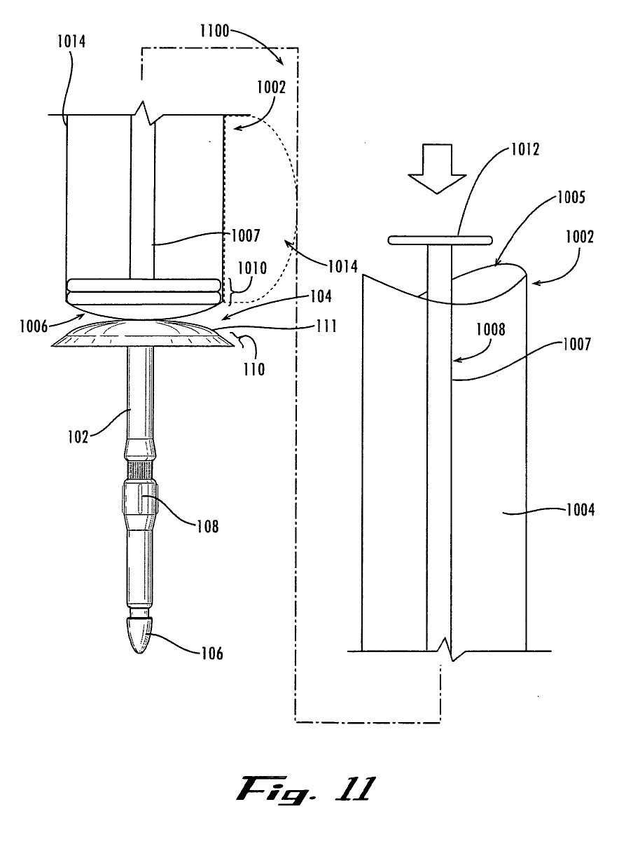

As the proximal operating portion 1012 is further depressed, the distal anvil advancing portion 1010 advances the compressed anvil through the second end 1006 of the tube as shown in FIG. 11. Once the anvil has been expelled out of the tube 1002, the anvil head member 104 moves to its deployed or expanded position as shown in FIG. 11.

In practice, a subject undergoing a gastric bypass procedure has the tube 1002 placed using techniques known in the art. For example, an appropriately sized tube 1002 is selected. The appropriate length of the tube 1002 can be measured from the tip of the subject's nose to the subject's xiphoid process. After an appropriate size and length tube 1002 is selected, the tube 1002, starting with the distal end, is inserted into the subject's mouth and advanced into the subject's stomach using routine methods known in the art. In one aspect, the second end of the tube is lubricated and inserted into the subject's mouth and advanced through the pharynx into the esophagus until the tube is seen pocketing in the gastric pouch with a laparoscopic camera. The tube can also be placed during other operations in a subject including, but not limited to, colo-colonic anastomosis during colon resections, small bowel resections and gastro-intestinal anastomosis. Appropriate size and length tubes can be selected by one skilled in the art.

The tube 1002 can be placed into the subject with the anvil located in its lumen 1004. The orogastric tube can also be placed into the subject without the anvil located in its lumen 1004. If the tube 1002 is placed without the anvil located in its lumen 1004, the anvil can be inserted into the lumen 1004 after placement of the tube 1002 in the subject. Similarly, the tube 1002 can be placed in the subject with or without the plunger 1008 located in its lumen 1004, and, if the tube 1002 is placed without the plunger 1008, the plunger can be subsequently inserted into the lumen 1004.

In one aspect, after placement of the tube 1002, the sizing balloon 1014 can be inflated for gastric pouch sizing. Conventionally, to form the gastric pouch, a stapler fires several rows of staples just distal to the margins of the sizing balloon 1014 and cuts between them. After the stapling and cutting is complete, the stomach has been divided into a gastric pouch and the remainder, or distal portion of the stomach. The creation of the gastric pouch can be accomplished laparoscopically.

The formed gastric pouch contains the distal end of the tube 1002 including the second end 1006. With the tube 1002 and its second end 1006 located in the gastric pouch the plunger 1008 can be advanced distally until it contacts the compressed anvil within the lumen 1004 of the tube 1002.

The second opening 1006 of the tube 1002 can be positioned in an overlying relationship to a portion of inner wall of the formed gastric pouch. As the plunger 1008 is advanced distally, the compressed anvil is expelled from the lumen 1004 through the second opening 1006 such that the distal shaft portion 106 exits first. As the anvil is expelled from the tube 1002, the distal shaft portion 1006 is advanced through the inner wall of the gastric pouch and into the abdominal cavity. The advancement of the distal shaft portion 106 through a wall of the gastric pouch can be visualized laparoscopically. The anvil can be advanced further distally so that increasing portions of the shaft member 102 extend out of the formed opening in the gastric pouch. The continued advancement can be accomplished by distal depression of the plunger 1008 or by pulling the anvil shaft member distally 102 from within the abdominal cavity.

As the anvil is advanced through the lumen 1004 of the tube and through a wall for the gastric pouch, the head member 104 is expelled from the lumen 1004 of the tube 1002. And, subsequently, the head member 104 moves from its compressed state to its expanded or deployed state. In use, in the deployed state, at least a portion of the shaft member 102 of the anvil, including the distal portion 106, extends through the wall in the gastric pouch. The deployed head member 104 is expanded and located within the interior of the formed gastric pouch.

A circular stapler can be connected with the shaft member 102 of the anvil as described above. Typically, during a gastric bypass procedure, the circular stapler is passed through and into the lumen of the subject's intestine prior to making a connection with the shaft member 102 of the anvil. Thus, the shaft member 102 of the anvil extends through the inner wall of the formed gastric pouch and through the intestinal wall and into the anvil shaft connecting member. Thus, in this aspect, intestinal and gastric pouch tissue are located between the staple forming surface 116 of the anvil head 104 and the staple firing surface of the circular stapler.

The circular stapler's staple firing surface is brought into registration with the staple forming surface 114 of the anvil with the intestinal and gastric pouch tissue located between the staple firing surface and the staple forming surface 114. Once in proper position and orientation, and with the tissue interposed, the circular stapler can be fired to advance staples through the interposed tissues and against the staple forming surface 114. The circular knife of the circular stapler can also be advanced inside the staple line (i.e., interior to staple forming surface 114), where it can cut the tissues located between the circular knife blade and the distal surface portion 112 of the head member 104. By firing the staples and cutting the tissue a passage between the gastric pouch and intestine is formed inside the staple line, which is the anastomosis made during gastric bypass surgery.

After the anastomosis is made, the circular stapler and the anvil are removed through the passage created inside the staple line. The plunger can also be retracted and removed from the lumen 1004 of the tube 1002 leaving the lumen substantially empty. In one aspect, once the lumen 1004 is substantially empty, an endoscope or other visualization device can be inserted into the first opening 1005 of the tube 1002 and can be passed distally through the lumen 1004. The endoscope can be advanced distally into the gastric pouch where the anastomosis can be visually evaluated.

Use of the system and anvil as described herein is not limited to gastric bypass procedures. The system can be used for anastomosis between any hollow organs for example, the described system can be used to place an anvil during other operations in a subject including, but not limited to, colo-colonic anastomosis during colon resections, small bowel resections and gastro-intestinal anastomosis.

Throughout this application, various publications are referenced. The disclosures of these publications in their entireties are hereby incorporated by reference into this application in order to more fully describe the compounds, compositions, devices and methods described herein.

The preceding description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. The corresponding structures, materials, acts, and equivalents of all means or step plus function elements in the claims below are intended to include any structure, material, or acts for performing the functions in combination with other claimed elements as specifically claimed.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Accordingly, those who work in the art will recognize that many modifications and adaptations to the present invention are possible and can even be desirable in certain circumstances and are a part of the present invention. Other embodiments of the invention will be apparent to those skilled in the art from consideration of the specification and practice of the invention disclosed herein. Thus, the preceding description is provided as illustrative of the principles of the present invention and not in limitation thereof. It is intended that the specification and examples be considered as exemplary only, with a true scope and spirit of the invention being indicated by the following claims.

What is claimed is:

1. A system for deploying an anvil for use with a surgical stapling device, comprising:
a tube having a first open end, a second open end and a lumen extending therebetween the first and second ends;
an anvil for use with the surgical stapling device, the anvil comprising a shaft member with a distal portion configured for engagement with the stapling device and a head member, wherein the head member is moveable between an intraluminal position and an extraluminal position, the intraluminal position being configured for slideable movement of the anvil thorough the tube lumen, and at least a portion of the extraluminal position being configured to form staples fired by the surgical stapling device after the anvil is expelled from the lumen through the second open end; and
a means for slideably moving the anvil though the lumen of the tube and for expelling the anvil through the second open end of the tube.

2. The system of claim 1, wherein the anvil advancing means comprises a plunger having a proximal operating portion, a distal anvil advancing portion and a shaft portion extending therebetween.

3. The system of claim 2, wherein the plunger is at least as long as the elongate length of the tube.

4. The system of claim 1, wherein the tube is in the range of about 20 French to about 50 French in size.

5. The system of claim 4, wherein the tube is in the range of about 20.0 centimeters (cm) to about 100.0 (cm) in length.

6. The system of claim 1, wherein the tube is an orogastric tube.

7. The system of claim 6, wherein the tube comprises a sizing balloon about its second open end for sizing a gastric pouch in a subject.

8. The system of claim 7, wherein the sizing balloon is inflatable with a volume of about 15 millileters (mL) to about 100 millileters (mL) of a gas or liquid.

9. The system of claim 1, wherein the head member is configured to move from its extraluminal position to its intraluminal position by deflection of the distal surface of the head member radially inward toward the shaft member of the anvil, and to move from its intraluminal position to its extraluminal position by deflection of the distal surface of the head member radially outward away from the shaft member of the anvil.

10. The system of claim 9, further comprising a means for biasing the head member from its intraluminal position to its extraluminal position.

11. The system of claim 9, wherein the anvil comprises material having shape memory such that the head is urged to move from its intralumial position to its extraluminal position based on the shape memory of the material.

12. The system of claim 1, wherein the head member is configured to move from its extraluminal position to its intraluminal position by deflection of the proximal surface of the head member radially toward the longitudinal axis of the shaft member of the anvil, and to move from its intraluminal position to its extraluminal position by deflection of the proximal surface of the head member radially away from the longitudinal axis of the shaft member of the anvil.

13. The system of claim 12, further comprising a means for biasing the head member from its intraluminal position to its extraluminal position.

14. The system of claim 12, wherein the anvil comprises material having shape memory such that the head is urged to move from its intralumial position to its extraluminal position based on the shape memory of the material.

15. The system of claim 1, wherein the head member is pivotally moveable relative to the longitudinal axis of the shaft member, and is configured to move from its extraluminal position to its intraluminal position by pivoting relative to the longitudinal axis of the shaft member, wherein, in the extraluminal position, the head member is substantially perpendicular to the longitudinal axis of the shaft member, and wherein, in the intraluminal position, the head member is generally parallel to the longitudinal axis of the shaft member.

16. The system of claim 1, wherein the head member has a diameter from about 20.0 millimeters (mm) to about 35.0 (mm) in its extraluminal position.

17. The system of claim 1, wherein the distal surface of the head member has a plurality of staple forming recesses defined thereon to form staples fired against the recesses by the surgical stapling device.

18. The system of claim 17, wherein the plurality of staple forming recesses are concentrically arranged thereon the distal surface and proximate to the periphery of the head member.

19. The system of claim 1, wherein at least a portion of the distal surface of the head member in its extraluminal position defines a tissue cutting surface for approximation with a blade of the surgical stapling device.

20. An anvil for use with a surgical stapling system having a surgical stapling device and a tube, comprising:
a shaft member with a distal portion configured for engagement with the stapling device; and
a head member, wherein the head member is moveable between an intraluminal position and an extraluminal position, wherein the head member is configured to move from its extraluminal position to its intraluminal position by deflection of the head member toward the longitudinal axis of the shaft member of the anvil, and to move from its intraluminal position to its extraluminal position by deflection of the head member away from the longitudinal axis of the shaft member of the anvil upon exit from the tube, wherein, in the extraluminal position, at least a portion of the head is configured to form staples fired by the stapling device, wherein, in the intraluminal position, the head of the anvil is configured for slidable movement therethrough the tube.

21. The anvil of claim 20, wherein the head member is configured to move from its extraluminal position to its intraluminal position by deflection of the distal surface of the head member radially inward toward the shaft member of the anvil, and to move from its intraluminal position to its extraluminal position by deflection of the distal surface of the head member radially outward away from the shaft member of the anvil.

22. The anvil of claim 21, further comprising a means for biasing the head member from its intraluminal position to its extraluminal position.

23. The anvil of claim 21, wherein the anvil comprises material having shape memory for its extraluminal position such that the head moves between its intralumial position and its extraluminal position based on the shape memory of the material.

24. The anvil of claim 20, wherein the head member is configured to move from its extraluminal position to its intraluminal position by deflection of the proximal surface of the head member radially toward the longitudinal axis of the shaft member of the anvil, and to move from its intraluminal position to its extraluminal position by deflection of the proximal surface of the head member radially away from the longitudinal axis of the shaft member of the anvil at a position proximal to the shaft member.

25. The anvil of claim 24, further comprising a means for biasing the head member from its intraluminal position to its extraluminal position.

26. The anvil of claim 24, wherein the anvil comprises material having shape memory such that the head is urged to move from its intralumial position to its extraluminal position based on the shape memory of the material.

27. The anvil of claim 20, wherein the head member is pivotally moveable relative to the longitudinal axis of the shaft member, and is configured to move between its extraluminal position and its intraluminal position by pivoting relative to the longitudinal axis of the shaft member, wherein, in the extraluminal position, the head member is substantially perpendicular to the longitudinal axis of the shaft member, and wherein, in the intraluminal position, the head member is generally parallel to the longitudinal axis of the shaft member.

28. The anvil of claim 20, wherein the head member has a diameter from about 20.0 millimeters (mm) to about 35.0 millimeters (mm) in its extraluminal position.

29. The anvil of claim 20, wherein the distal surface of the head member has a plurality of staple forming recesses defined thereon to form staples fired against the recesses by the surgical stapling device.

30. The anvil of claim 29, wherein the plurality of staple forming recesses are concentrically arranged thereon the distal surface and proximate to the periphery of the head member.

31. The anvil of claim 20, wherein at least a portion of the distal surface of the head member in its extraluminal position defines a tissue cutting surface for approximation with a blade of the surgical stapling device.
